(19) United States
(12) Patent Application Publication
(10) Pub. No.: US 2021/0000650 A1
(43) Pub. Date: Jan. 7, 2021

(54) EVENT-TRIGGERED EYE OCCLUSION

(71) Applicant: Focus Labs, LLC, St. Paul, MN (US)

(72) Inventors: Nicholas Roseth, St. Paul, MN (US); Darrin Swagel, Minnetonka, MN (US)

(21) Appl. No.: 16/918,745

(22) Filed: Jul. 1, 2020

Related U.S. Application Data
(60) Provisional application No. 62/869,414, filed on Jul. 1, 2019.

(51) Int. Cl. A61F 9/08; G02B 27/00; G02B 27/01; G02B 5/30

(52) U.S. Cl. CPC A61F 9/08; G02B 27/0075; G02B 7/023; G02B 5/3016; G02B 2027/0178; G02B 27/017

(57) ABSTRACT

A system and method for user-initiated occlusion of vision includes an electrically or electromagnetically controlled lens disposable in front of one of the user's eyes. The lens may be controlled by a trigger device including a sensor that may be activated by various motions of the user's body. The trigger device may communicate with the lens wirelessly. The lens may be incorporated in a wearable apparatus or head gear having a variety of forms.

### RELATED APPLICATIONS

This application claims priority to U.S. Provisional Application No. 62/869,414 filed on Jul. 1, 2019, the contents of which are hereby incorporated by reference in its entirety.

### TECHNICAL FIELD

Embodiments described herein generally relate to wearable optics and, more particularly, to eyewear that can assist a user with various hand-eye coordination tasks such as those involved in the aiming of firearms, use of sports equipment, tools, or the like through the use of a variably shaded lens that partially occludes the individual's dominant eye. The user utilizes a trigger mechanism to initiate the immediate transition of the lens from a clear state to a full or partially shaded state, and vice versa, on demand and when needed.

### BACKGROUND

When a person's dominant eye is on the side of the body that is opposite his or her dominant hand, that person is said to experience a cross-dominance eye condition, also known as mixed laterality. When such a person is required to establish an accurate line of sight using the non-dominant eye, the result is often an inaccurate and ineffective line of sight.

Tools and equipment are often constructed with the presupposition that the user's dominant eye will be on the same side of the body as the user's dominant hand. However, when the user's eyesight is impacted by cross-dominance, that user's level of performance is hampered in circumstances where an accurate line of sight must be established. That situation occurs because the dominant eye often supersedes the non-dominant eye that is being used to establish the line of sight, with the result that the user's perceived line of sight is skewed. To prevent that from happening, the user often closes, or otherwise obscures, the vision of the dominant eye so that the non-dominant eye is temporarily forced to become the dominant eye.

Also, there are many activities where focus and aiming may be improved by fully or partially occluding vision in one eye, even if the user does not experience cross-dominance.

In activities where one eye is used to handle a sighting or aiming function (such as in aiming of firearms, bows, or other sporting implements, for instance), the act of closing or totally obscuring the vision of the dominant eye does improve the individual's ability to establish an accurate line of sight. However, the act of closing one eye also degrades that person's peripheral vision and depth perception which are two functions of human binocular vision that are very helpful and conducive to an accurate aiming process.

### SUMMARY

A system and method for user-initiated occlusion of vision includes an electrically or electromagnetically controlled lens mounted in front of one of the user's eyes. The lens may be controlled by a trigger device including a sensor that may be activated by various motions of the user's body. For example, a trigger device may include a sensor or monitoring system for detecting activation of a pushbutton or activation of a pressure switch, as well as reading a load cell, strain sensor, touch sensor, proximity sensor or deflection sensor. The trigger device may communicate with the lens wirelessly or through a wired connection. The lens may be incorporated in a wearable apparatus or head gear having a variety of forms.

An embodiment of a wearable apparatus includes a lens controllable to selectively occlude vision; a housing enclosing a controller and coupled to the lens, the housing adapted to be worn by a user so that the lens is positioned in front of a user's eye; and a user-activated trigger mechanism adapted to transmitting a signal to the controller when activated by the user, the controller causing the lens to occlude the user's eye.

In a further embodiment, an occlusion system includes a lens controllable to temporarily occlude vision; a housing; a double hinge coupling the lens to the housing, the double hinge flexibly positioning the lens in front of a user's eye; a mounting structure removably coupling the housing to a device worn on a head of the user; a controller for receiving a signal to cause the lens to occlude a portion of a field of vision of the user's eye; and a user-activated trigger mechanism comprising a sensor for detecting an action of the user, generating the signal to occlude vision and transmitting the signal to the controller.

In another embodiment, an occluder for temporarily occluding a user's vision in one eye in response to a user-activated event when mounted to a device worn on a user's head, includes an electrically-controllable lens; a housing adapted to be mounted to the device so that the electrically-controllable lens is positioned in front of either of a user's eyes; a double hinge coupling the electrically-controllable lens to the housing, the double hinge flexibly positioning the electrically-controllable lens in front of a user's eye; a controller for receiving a signal to cause the electrically-controllable lens to occlude a portion of a field of vision of the user's eye.

In either of the above embodiments, the signal is transmitted wirelessly.

In any of the embodiments, the mounting structure is adapted to attach the housing to either arm of a pair of glasses. Further, the double hinge may enable the lens to be oriented relative to a proximal lens of the pair of glasses.

In any of the above embodiments, the lens is electrically or electromagnetically controlled to temporarily occlude vision. Further, the lens may include an electrically-controllable liquid crystal display (LCD) or a microelectromechanical systems (MEMS) shutter array.

In embodiments, the controller causes the lens to be occluded for a selectable degree of opacity. In further embodiments, the controller causes the lens to be occluded for a selectable period of time or after a selectable delay.

In any of the embodiments, the user-activated trigger mechanism further comprises a pressure sensor. Further, the user-activated trigger mechanism may be incorporated in a glove. Yet further, the user-activated trigger mechanism may include a membrane switch.

In any of the embodiments, the mounting structure may be adapted to be attached to a headband, headset or any type of headgear.

### BRIEF DESCRIPTION OF THE DRAWINGS

Illustrative embodiments of the disclosed technology are described in detail below with reference to the attached drawing figures, which are incorporated by reference herein and wherein:

FIGS. 1A and 1B are perspective views of an event-initiated occluding device mounted to a pair of glasses, in an embodiment.

FIG. 2 is a perspective view of the occluder of FIGS. 1A and 1B, in an embodiment.

FIG. 3 is an exploded view of the occluder of FIG. 2, in an embodiment.

FIGS. 4A-4C are top views of the occluder of FIG. 3 illustrating several lens positions, in embodiments.

FIG. 5 shows the occluder and mounting structure of FIGS. 1A-1B, in embodiments.

FIGS. 6A-6C are side views illustrating positioning of the occluder relative to the mounting structure of FIG. 5, in embodiments.

FIG. 7 is an exploded view of the mounting structure of FIGS. 1A-1B, in an embodiment.

FIGS. 8A and 8B are perspective views illustrating installation of the mounting structure of FIG. 7 on a pair of glasses, in embodiments.

FIG. 9 is a perspective view of a user-activated trigger mechanism, in an embodiment.

FIG. 10 is an exploded view of the user-activated trigger mechanism of FIG. 9, in an embodiment.

FIG. 11A is a perspective view of a mounting structure, in an embodiment.

FIG. 11B is an exploded view of the mounting structure of FIG. 11A.

FIGS. 12A and 12B are perspective views of the occluder of FIG. 2 and the mounting structure of FIG. 11A attached to a pair of glasses.

FIGS. 13A and 13B are perspective views of the occluder of FIG. 2 in use with a headband, in embodiments.

FIG. 14A is a side view of the headband of FIGS. 13A and 13B as it would be positioned on a user's head, in embodiments.

FIG. 14B is a perspective view of FIG. 14A.

FIGS. 15A and 15B are perspective views of the occluder of FIG. 2 in use with a headset, in embodiments.

FIG. 16 is a side view of occluder 102 and the headset of FIGS. 15A and 15B.

FIG. 17 is a perspective view of the headset of FIGS. 15A and 15B as it would be positioned on a user's head.

FIGS. 18A-18B are perspective views of an event-initiated occluding device mounted to a pair of glasses with a bayonet mounting structure, in embodiments.

FIG. 19A is a perspective view of an occluder and the bayonet mounting structure of FIGS. 18A-18B, in embodiments.

FIG. 19B is an exploded view of the bayonet mounting structure of FIG. 19A, in embodiments.

FIG. 20 is a horizontal cross-section of the occluder and bayonet mounting structure of FIG. 19A, in embodiments.

FIG. 21 is a vertical cross-section of the occluder and bayonet mounting structure of FIG. 19A, in embodiments.

FIGS. 22A-22B are perspective views of an event-initiated occluding device mounted to a pair of glasses with a bayonet clip mounting structure, in embodiments.

FIG. 23 is a perspective view of the occluder and bayonet clip mounting structure of FIGS. 22A-22B, in embodiments.

FIG. 24 is perspective view of the bayonet clip mounting structure of FIG. 23, in embodiments.

FIG. 25 is a block diagram of a circuit board for use in an occluder, in embodiments.

FIG. 26 is a perspective view of an elastic strap mounting structure, in embodiments.

### DETAILED DESCRIPTION

While the invention is amenable to various modifications and alternative forms, specifics thereof have been shown by way of example in the drawings and will be described in detail. It should be understood, however, that the intention is not to limit the invention to the embodiments described.

In the following description, some specific details are included to provide a thorough understanding of various disclosed embodiments. One skilled in the relevant art, however, will recognize that embodiments may be practiced without one or more of these specific details, or with other methods, components, materials, etc. In other instances, well-known structures or circuitry associated with sensing transducers and circuitry, communication circuitry, optical systems, and control systems have not been shown or described in detail to avoid unnecessarily obscuring descriptions of the embodiments of the present systems, methods and apparatus.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a referent feature, structure, or characteristic described regarding the embodiment is included in at least one embodiment. Thus, the appearances of the phrases “in one embodiment,” or “in an embodiment,” or “another embodiment” in various places throughout this specification are not necessarily all referring to the same embodiment. Furthermore, the features, structures, or characteristics may be combined in any suitable manner in one or more embodiments.

It should be noted that, as used in this description, the singular forms “a,” “an,” and “the” include plural referents unless the content clearly dictates otherwise. It should also be noted that the term “or” is generally employed in its sense including “and/or” unless the content clearly dictates otherwise. Furthermore, numbered lists appearing herein do not imply any limit, closed set, ordering, or priority.

Aspects of the embodiments recognize that, when a person with cross-dominance vision has both eyes open, his or her depth perception and peripheral vision are fully functional. Further, when that person's non-dominant eye is momentarily forced to act as the dominate eye, he or she would be able to establish an accurate line of sight using the non-dominant eye. However, forcing use of a non-dominant eye by simply closing the dominant eye results in loss of depth perception and peripheral vision, for example. Some of the embodiments described herein enable a user to simultaneously maintain depth perception and peripheral vision while concurrently causing the non-dominant eye to temporarily become dominant during the critical period of the aiming process.

One aspect of the embodiments is that a user with cross-dominant vision can utilize his or her binocular vision while concurrently being able to establish an accurate line of sight (as in aiming a rifle) using the non-dominant eye. The embodiments described herein achieve this goal by rapidly and dynamically causing the dominant eye to be partially occluded at a critical moment. This action temporarily forces the non-dominant eye to perform as the dominant eye, while still enabling the dominant eye to remain open and at least partially usable most of the time during the aiming activity, thereby providing the user with the full benefits of binocular vision.

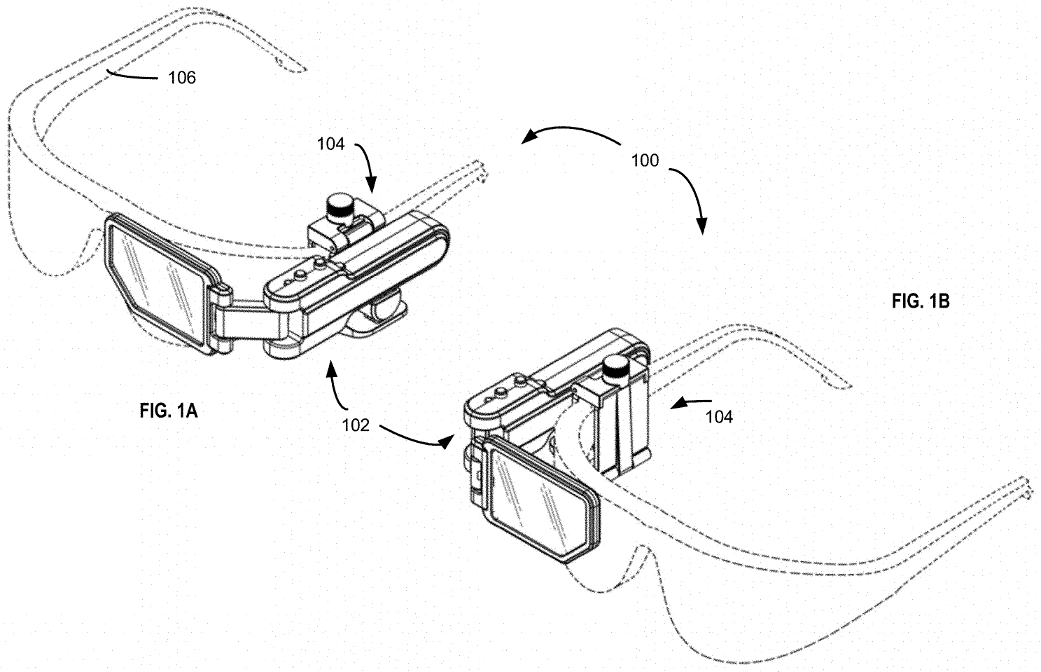

According to some embodiments, a wearable device is provided with a form factor of eyeglasses, as shown in FIGS. 1A and 1B. In this embodiment, the glasses are fitted with an occluding device positioned in front of a user's eye. The occluding device is transparent in a default state and selectively occludes a user's eye in response to a triggering event.

FIGS. 1A and 1B are perspective views of occluding device 100, in embodiments. Occluding device 100 generally includes occluder 102 which is attached to a pair of glasses 106 by mounting structure 104. In FIG. 1A, occluder 102 and mounting structure 104 have been mounted on the left arm of glasses 106 so they may be used to occlude the left eye. In FIG. 1B, occluder 102 and mounting structure 104 have been mounted on the right arm of glasses 106. Both occluder 102 and mounting structure 104 are designed so they may be mounted on either arm as shown and described in more detail below.

Although occluding device 100 is shown in FIGS. 1A and 1B as a separate device mounted to a pair of glasses, in embodiments, occluding device 100 may be incorporated within the structure of glasses 106 themselves. Further, different mounting structures 104 may be used. Occluding device 100 may be mounted to a helmet, headband, or any type of headgear that allows occluding device 100 to be maintained in a position in front of a user's non-dominant eye.

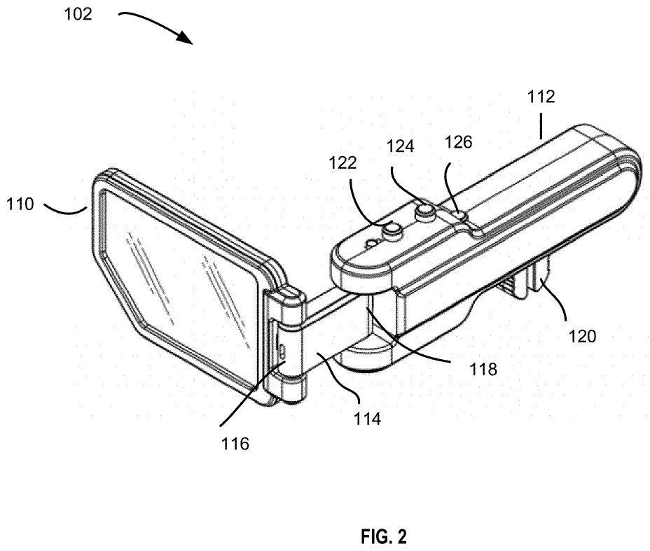

FIG. 2 is a perspective view of occluder 102 of FIGS. 1A and 1B. Occluder 102 includes lens 110 and housing 112 flexibly connected to each other by double hinge 114. Double hinge 114 is connected to both lens 110 by means of pin 116 and housing by means of pin 118 in such a way as to provide a large degree of flexibility in positioning lens 110, described in more detail below. Further flexibility in positioning lens 110 is provided by pivot 120, which interacts with mounting structure 104 to provide a pitch adjustment for lens 110. Housing 112 may be a lightweight, portable structure for enclosing electrical components used to operate occluder 102. For example, housing 112 may contain a controller or processor, power supply and a receiver for receiving a control signal. Control buttons 122, 124 and 126 are provided for controlling operation of occluder 102, such as turning it on and off, or selecting operational features such as the degree or duration of light reduction provided by lens 110.

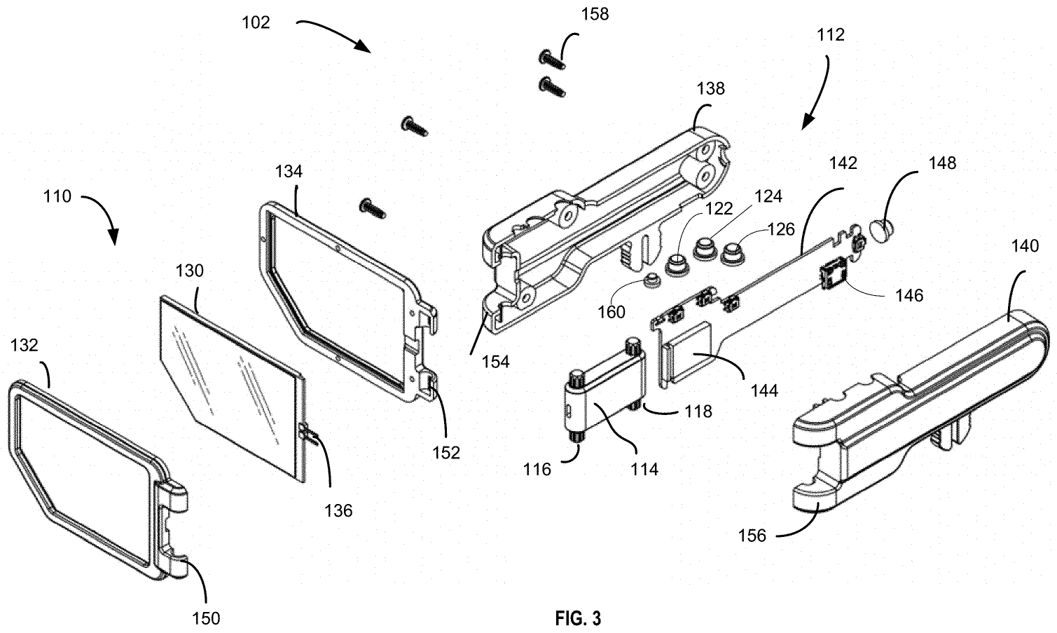

FIG. 3 is an exploded view of occluder 102 of FIG. 2. Lens 110 includes shutter 130, which is enclosed by lens front frame 132 and lens back frame 134. Shutter 130 may be, for example, an electrically-controllable liquid crystal display (LCD) that, when triggered, rapidly obstructs or partially blocks light transmission through the lens. In embodiments, an LCD includes a coating that changes in perception of transparency depending on the angle of incidence of light striking the eye. For example, the coating causes a user to perceive the area directly in front of the eye to approximately 20-30 degrees to be completely opaque. However, areas farther towards the periphery of the eye perceive some lightness and therefore, visibility. In embodiments, occluder 102 may also be controlled to preset a preferred degree of darkening of an LCD. This is to allow lens 110 to be attuned to the vision of particular users, who have varying sensitivities to light and varying degrees of cross-dominance. The degree of opacity achieved by an LCD is controlled by the voltage provided.

In embodiments, shutter 130 may also be a microscale shutter array constructed using microelectromechanical systems (MEMS), microscale or nanoscale blinds, an image sensor-display system that momentarily interrupts a display or camera or other light-transmission disruptive technology to achieve the occlusion effect, for example. In embodiments, frames 132 and 134 encircle a transparent, shatter-resistant layer of glass or polycarbonate, for example. These layers enhance durability and provide protection for a user's eye.

In embodiments, shutter 130 is operable to allow a user's view to become fully or partially occluded rapidly, in other words, faster than a typical human reaction time. In some examples, the occluder achieves effective occlusion in less than 300 ms. In other examples, occlusion is achieved in less than 200 ms, less than 100 ms, or less than 50 ms. Further, shutter 130 may be controlled so that only a central portion of shutter 130 is occluded instead of the entire shutter. The central portion may be positioned such that it is aligned with a barrel of a shotgun or other sighting mechanism when the wearer is engaged with the firearm or instrument. Control signals may be provided to shutter 130 through leads 136.

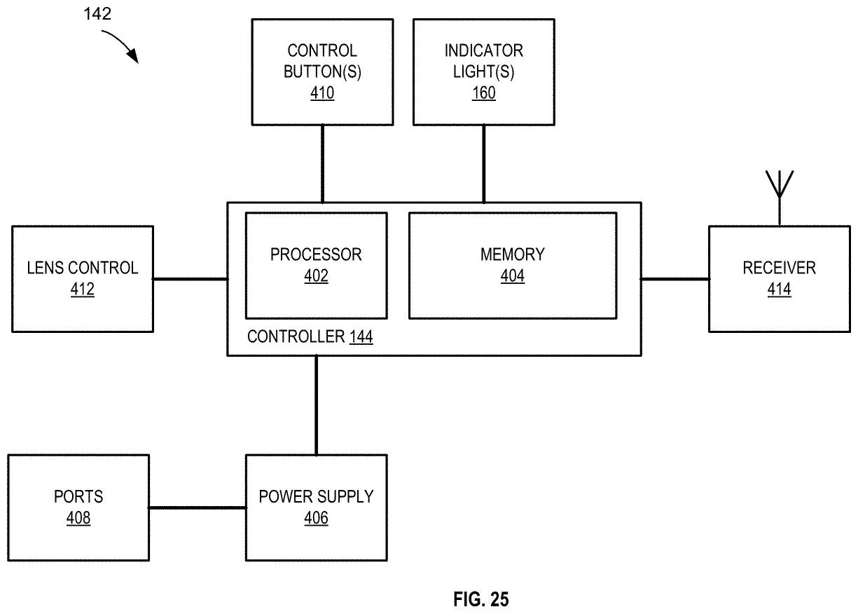

Housing 112 includes side shells 138 and 140 that enclose circuit board 142. A block diagram of components mounted on circuit board 142 is shown in FIG. 25. Various functional components are shown in FIG. 25 with various interconnections, however, other arrangements and interconnections are possible. The functions disclosed herein may be combined in different components and interconnected in different ways, in embodiments. Controller 144 is mounted on circuit board 142 for controlling operation of occluder 102. Controller 144 includes a processor 402 and a memory 404 for storing instructions for execution by processor 402. Power supply 406 may also be mounted on circuit board 142. A power supply may include one or multiple batteries, which may be disposable batteries or a rechargeable battery. One or more ports 408 may be provided. For example, USB charging port 146 provides for a charging cable to be connecting to housing 112 so that power supply 406 may be charged by connecting occluder 102 to a wall outlet or other source of power. Other ports, such as DC or AC ports, may be provided.

One or more control buttons or switches 410 may be provided for interacting with controller 144 to operate occluder 102. Button 148 of FIG. 3 may be used to power occluder 102 on or off during use. Buttons 122, 124 and 126 of FIG. 2 may provide the functions of a master on/off power button, a radio frequency button to select the radio frequency that activates the occluder 102, a selector switch to select the degree of light reduction, or opacity that occluder 102 may provide, a selector switch to preset an elapsed time period for maintaining occlusion and a timed interval or delay selection switch, for example. In embodiments, any of buttons 122, 124, 126 and 148 may be placed in other locations on housing 112.

In embodiments where occluder 102 is integrated into a glasses frame, a lens selection switch may be provided to select which single lens should be occluded. Housing 112 may also include one or more indicator lights 160 for alerting a user to an operational state, such as a battery charge indicator or a pairing indicator. Side shells 138 and 140 may be secured by means of screws, represented at 158, for example. Side shells 138 and 140 may also be secured by glue, welds or other attachment means.

Circuit board 142 may also include one or more components as lens control 412 for providing a control signal to lens 110. Receiver 414 receives a control signal from a trigger mechanism, described in more detail below, to control lens 110.

Lens 110 and housing 112 are connected by means of double hinge 114. Pin 116 of double hinge 114 is inserted into a receiving socket formed by tabs 150, 152 of lens front frame 132 and lens back frame 134, respectively. Lens 110 may rotate axially around the vertical axis of pin 116. Pin 118 of double hinge 114 is inserted into a receiving socket formed by projections 145 and 156 of side shells 138 and 140, respectively. Double hinge 114 may rotate axially around the vertical axis of pin 118.

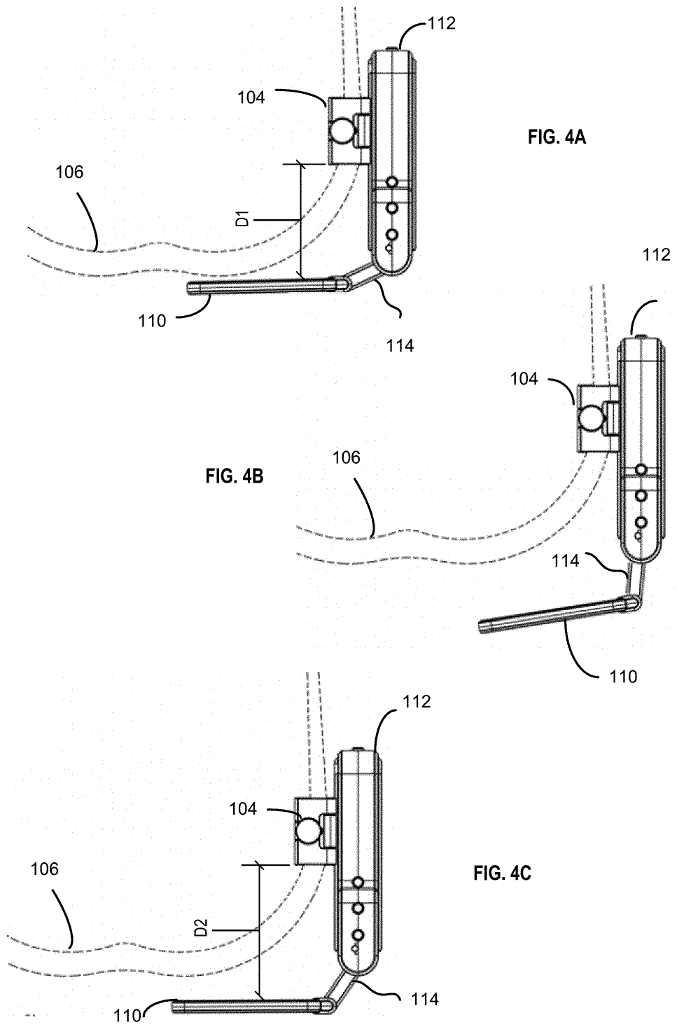

FIGS. 4A-4C are top views of the occluder of FIG. 3 illustrating several lens positions, in embodiments. In all three figures, housing 112 is attached to mounting structure 104 which has been installed on glasses 106. In FIG. 4A, double hinge 114 is positioned so that lens 110 is generally parallel to a lens of glasses 106 and separated by a distance D1 from mounting structure 104. In FIG. 4B, double hinge 114 and lens 110 have been rotated to move lens 110 out and away from glasses 106. In FIG. 4C, lens 110 has been rotated back towards glasses 106 relative to its position in FIG. 4B so that it is again generally parallel to a lens of glasses 106, but is at a distance D2 from mounting structure 104, where D2>D1. Distances D1 and D2 are intended only to show the relative positioning of lens 110.

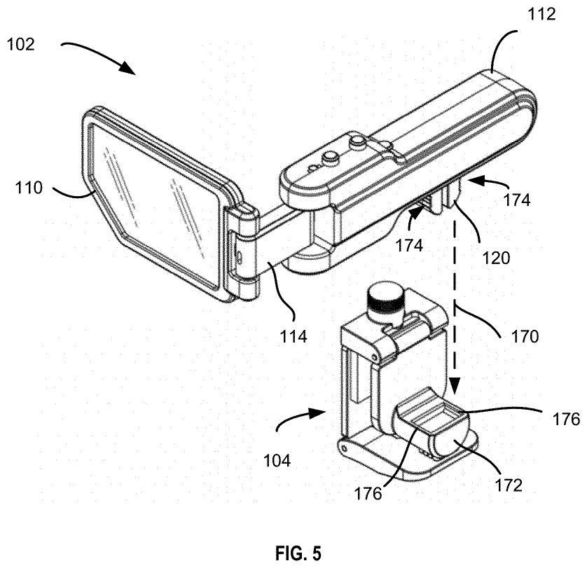

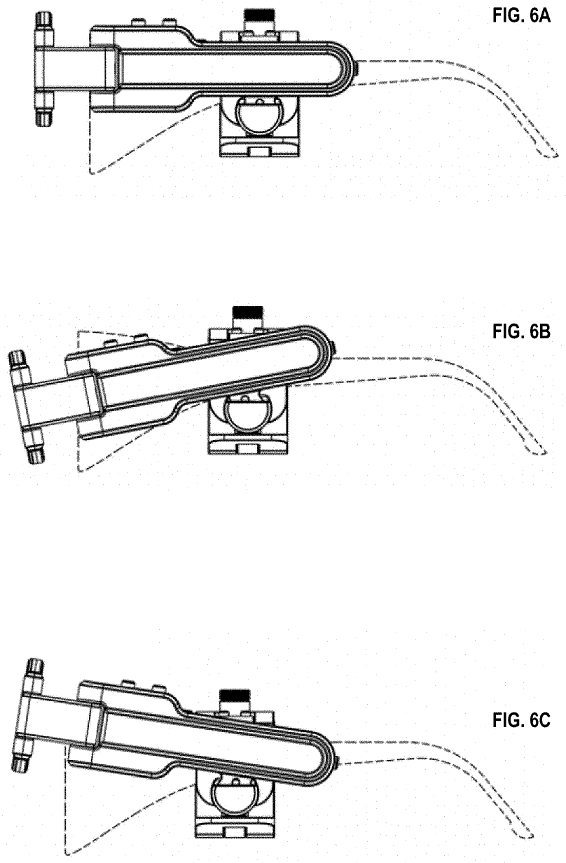

FIG. 5 is shows the occluder and mounting structure of FIGS. 1A-1B, in embodiments. FIGS. 6A-6C show side views of occluder 102 relative to mounting structure 104. FIGS. 5 and 6A-6C are best viewed together in the following discussion. Occluder 102 includes pivot 120 on a lower surface of housing 112. As shown in FIG. 5, the illustrated pivot 120 is comprised of two downwardly projecting members. When attaching occluder 102 to mounting structure 104, pivot 120 is inserted into cup 172 on mounting structure 104 using a snap or friction fit as shown by dotted line 170. After pivot 120 is inserted into cup 172, a pitch adjustment may be made to occluder 102 by tilting it around a horizontal axis through the cup 172 as shown in FIGS. 6A-6C. In FIG. 6A, occluder 102 is generally horizontal and parallel to the glasses frame. In FIG. 6B, occluder 102 is tilted down while in FIG. 6C, it is tilted up. Occluder 102 is retained in one of the positions of FIGS. 6A-6C by means of detents 174 in pivot 120 engaging with ridges 176 in cup 172. The gap between downwardly projecting members of the pivot 120 enables a degree of inward deflection. As a user tilts occluder 102, detents 174 are engaged with ridges 176, depending on the orientation of occluder 102. Mounting structure 104 and occluder 102 are left/right reversible for attachment to either side of a user's eyeglasses. Occluder 102 is provided with a pitch adjustment so that the user can modify the distance between the occluding lens of the embodiment and the lens of the user's safety or prescription glasses; thusly accommodating the curvature variances of prescription and safety eyeglass lenses. Double hinge 114, as explained above, also allows adaptation of occluding device 100 to a variety of eyewear sizes and configurations.

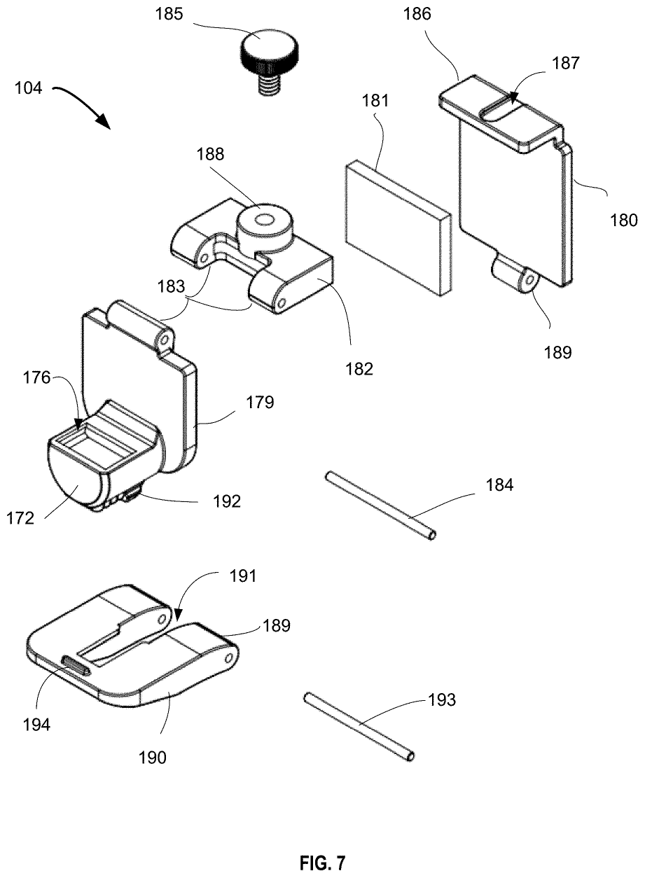

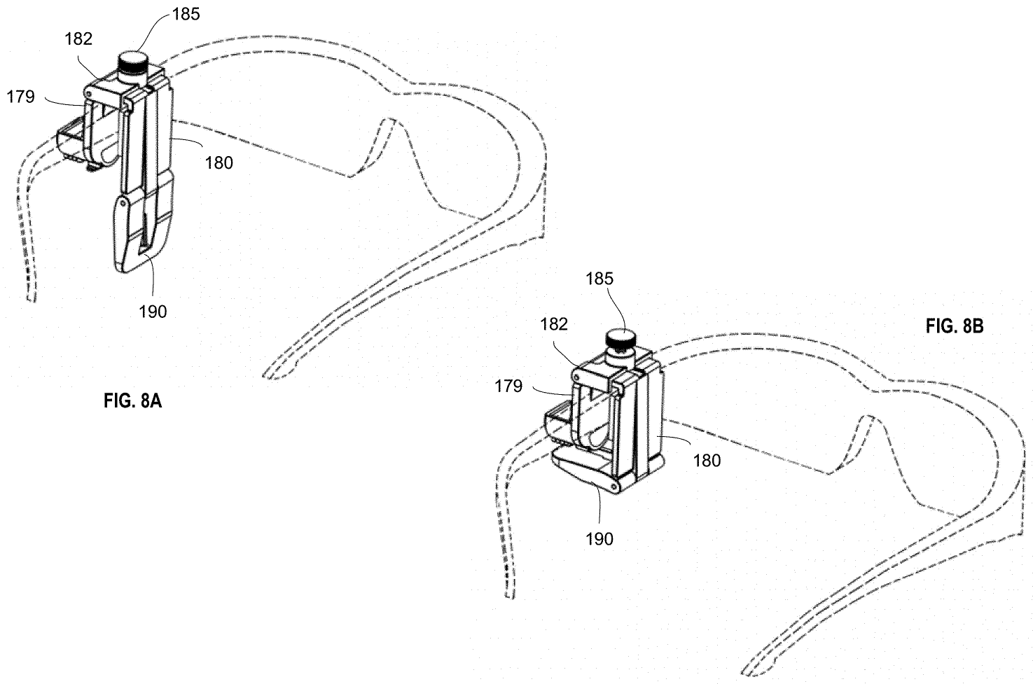

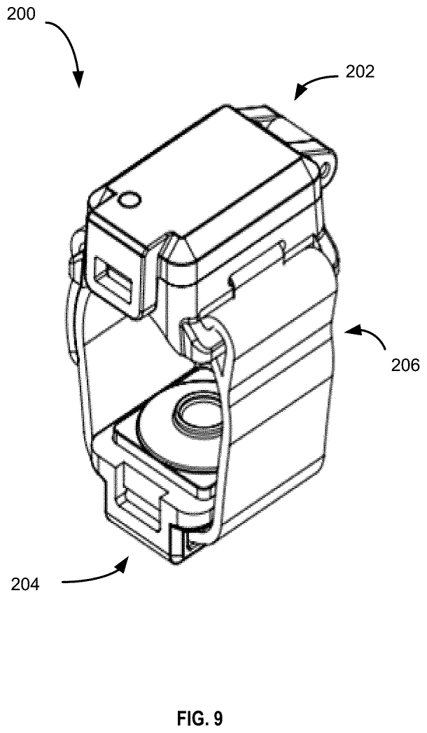

FIG. 7 is an exploded view of the mounting structure of FIGS. 1A-1B, in an embodiment. FIGS. 8A and 8B are perspective views illustrating installation of the mounting structure of FIG. 7 on a pair of glasses, in embodiments. FIGS. 7, 8A and 8B are best viewed together in the following description. Mounting structure 104 may be flexibly mounted on either arm of a wide variety of pairs of glasses. As shown in FIG. 5, cup 172 includes ridges 176 for engaging with pivot 120 of occluder 102. Cup 172 is attached to a lower edge of outer face 179. Outer face 179 and inner face 180 may be positioned on either side of an arm of a pair of glasses, in the vicinity of the temple of a user. In embodiments “outer” and “inner” used relative to a user's head in that inner face 180 is closer to the head and outer face 179 is farther from the user's head. Mounting structure 104 may be mounted to either arm of a pair of glasses. Padding 181 may be foam or some other flexible material so as to help secure mounting structure 104 to the arm of the pair of glasses so it doesn't shift during use. Outer face 179 and inner face 180 are attached to each other by upper bracket 182. Outer face 179 may pivot relative to upper bracket 182 by means of hinge 183 and pin 184.

Inner face 180 is substantially parallel to outer face 179. Inner face 180 also includes upper projection 186 extending out generally perpendicularly from an upper edge of inner face 180. When attaching mounting structure 104 to a pair of glasses, upper projection 186 is inserted into upper bracket 182 while outer face 179 and inner face 180 are brought together with an arm of the glasses between them. When a secure fit is achieved, screw 185 is inserted through fitting 188 to engage with slot 187 in upper projection 186 to secure outer face 179 and inner face 180 at a certain distance from each other.

Inner face 180 is attached to lower bracket 190 by means of hinge 189 and pin 193. As mounting structure 104 is placed over the arm of a pair of glasses, lower bracket 190 is in a generally open position, as shown in FIG. 8A. In embodiments, lower bracket 190 may be placed in any position that allows the arm of the glasses to be inserted between outer face 179 and inner face 180. After screw 185 is secured, lower bracket 190 is brought up against cup 172 so that slot 191 and tab 194 engage with fitting 192 in a snap fit, as shown in FIG. 8B.

Occluder 102 causes occlusion in response to a control signal generated by a triggering event, according to embodiments. A triggering event is detected by a sensor or monitoring system, which may be remotely situated from occluder 102. The sensor or monitoring system may be specifically constructed to fit and work with one or more specific applications. Various event types may be accommodated according to corresponding embodiments.

In some examples, a triggering event includes an aiming process of a shooting implement. The aiming-process event may be detected based on positioning or motion of the shooting implement, pressure applied to some part of the shooting implement (e.g., the stock, handle, trigger, or the like), release of a safety mechanism, cocking of a hammer, or some combination of these occurrences. In other examples, a triggering event may include a human-device interaction, such as a swinging motion (e.g., the start of a batter's swing of a baseball bat, a golfer's backswing or transition between backswing and forward swing, an approach of a human observer's eye towards an optical instrument or viewfinder, tensioning of a bow, grasping of a device in an operative fashion, or the like).

For detecting one or more of these occurrences, a suitable sensor or monitoring system may include one or more sensing technologies such as detection of activation of a pushbutton, detection of activation of a pressure switch, reading of a load cell, reading of a strain sensor, reading of a touch sensor, reading of a proximity sensor, reading of a deflection sensor, or the like.

The sensor or monitoring system may be communicatively coupled to the occluder 102 via a wired or wireless coupling. Detection of a triggering event may cause a triggering indication to be communicated over the wired or wireless interface from the sensor or monitoring system to occluder 102. The wired or wireless coupling may utilize any suitable signaling technology, such as digital-logic signaling, baseband coding, packet-based communications, etc. Wireless communications may be achieved with RF, infrared, ultrasonic, or some variation of any of these modalities. In some embodiments, a personal-area network such as Bluetooth® low energy (BLE), near-field communications (NFC), or similar standardized or non-standardized networking technology may be utilized. In other embodiments, a dedicated transmitter is provided on the side of the sensor or monitoring system, and a dedicated receiver is provided in occluder 102.

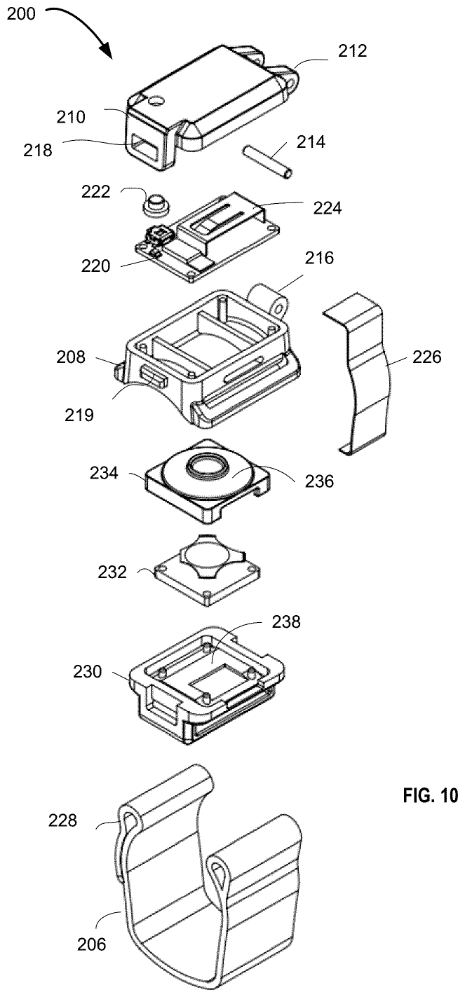

FIG. 9 is a perspective view of an embodiment of a sensor or monitoring system in the form of a user-activated trigger mechanism 200. FIG. 10 is an exploded view of the user-activated trigger mechanism of FIG. 9, in an embodiment. FIGS. 9 and 10 are best viewed together in the following description. Trigger mechanism 200 may be worn on a user's finger, either is a standalone ring-type device or as part of a full or partial glove. Generally, trigger mechanism 200 includes an electronics case 202, switch case 204 and attachment mechanism 206, for example, a strap or other device for attaching trigger mechanism 200 to a user's finger.

Electronics case 202 includes base 208 and lid 210. Lid 210 is attached to base 208 by means of barrel hinge 212, 216 and pin 214. To retain lid 210 in a closed position, tab 219 in base 208 may snap into opening 218 in lid 210. Electronics for controlling the operation of trigger mechanism 200 and a transmitter (not shown) for sending a control signal are mounted on circuit board 220, which may also include, for example, a control button 222 and a clip 224 for retaining a battery. A transmitter may use a variety of wireless protocols, including RF, ultrasonic, optical. Electronics case 202 is coupled to switch case 204 by wiring harness 226, which may be attached to or secured within attachment mechanism 206. The circumference of attachment mechanism 206 may be adjusted by means of clasp 228, which may include hook-and-loop tape or other devices for adapting attachment mechanism securely to a user's finger.

Switch case 204 includes a base 230, sensor 232 and button 236 and lid 234. Sensor 232 is mounted securely within recessed area 238 then covered by lid 234. In embodiments, button 236 may be made of a flexible material so that pressure from a user's finger against a solid surface activates sensor 232 to send a signal to occluder 102 over the transmitter in electronics case 202. In embodiments, sensor 232 may be selected from many different devices. For example, sensor 232 may be a button that generates an electrical signal when manually depressed by the user such as a membrane switch that detects when one electrical contact meets another electrical contact to complete a circuit. Sensor 232 may also be a pressure sensor that elicits an electrical signal in response to the occurrence of a predetermined pressure load upon the device (e.g., load cell, etc). Sensor 232 may also be a proximity sensor that employs capacitive, inductive, or RF-propagation (e.g., Bluetooth) technology, an individual or combination of sensors where one sensor triggers a second sensor based on the proximity of the two sensors to each other.

Other types of triggering mechanisms may be used with occluder 102. For example, an accelerometer may be attached to a piece of equipment such as a shotgun or bow. When the equipment with the accelerometer is accelerated to a specified speed or along a certain trajectory or detectable movement pattern, the triggering event is detected. An optical subsystem with a laser emitter and deflection detector may also be used. An optical subsystem operates by reflecting the laser off a surface (e.g., the back side of a gun's trigger), with the reflection impinging on the deflection detector. Movement of the laser along the deflection detector indicates angular motion of the reflecting surface (e.g., trigger).

A triggering switch may be embedded inside of an article worn by the user or attached to an object such as a shotgun, bow, or golf club. When pressure is applied to the triggering button, a signal is sent that activates occluder 102 and causes it to darken.

A strap-on shoulder recoil pad with an embedded pressure sensor may be used as a trigger mechanism. In this embodiment, a recoil-reducing shoulder pad worn by the shooter is equipped with a pressure sensitive triggering device. Pressure is applied to the device when the gun is shouldered and readied to fire, thereby activating the transmitter to send a signal that causes occluder 102 to darken.

A trigger mechanism may be embedded in a removable gun butt recoil pad that is friction fitted by the user over the butt end of the rifle or shotgun stock. When the gun is pressed to the shooter's shoulder and readied for firing, the applied pressure activates the embedded trigger, sending a signal that causes occluder 102 to darken. In addition to the triggering device, the gun butt recoil pad is also equipped with various other electrical components.

A partial finger glove may be worn by the user and connected to a wrist band that houses various electrical components. When the user presses his or her finger against any solid surface, the triggering device sends a signal to activate occluder 102. In shooting, the finger glove would typically be worn on the hand that cradles the forestock of a rifle or shotgun.

A shooting glove worn by the user may include a trigger mechanism in the index finger, the middle finger, or the ring finger of the glove. When the user presses the triggering device against any solid surface, the triggering device sends a signal to activate the occluder. The glove wrist strap contains various electrical components; a wire line connects the electrical components to the triggering mechanism in the finger of the glove.

A ring 200 as describe above may be used as a trigger mechanism: worn like a typical finger ring, the ring would be configured to: a) be a pressure sensitive triggering button that would be activated when pressed against the surface of the gun; or b) be a magnetically activated triggering button that is activated upon contact with a steel surface such as a gun barrel. In each embodiment, the triggering device will cause a signal to be initiated that will subsequently make occluder 102 darken.

In another embodiment, a trigger mechanism may be attached to a gun stock. A strap may be affixed to a shotgun or rifle that contains a triggering button and various electrical components. When the shooter presses the triggering button, a signal would be initiated that causes occluder 102 to darken. The strap may be affixed on a rifle, shotgun, or bow in the location preferred by the user.

In a further embodiment, the sensor or monitoring system of a trigger mechanism provides an indication of cessation of a triggering event. This event-cessation indication may include a separate signal or packet that is distinct from the triggering signal, or it may incorporate the triggering signal (e.g., cessation of a continuous signal that starts in response to the triggering event and is maintained throughout the occurrence of the triggering event).

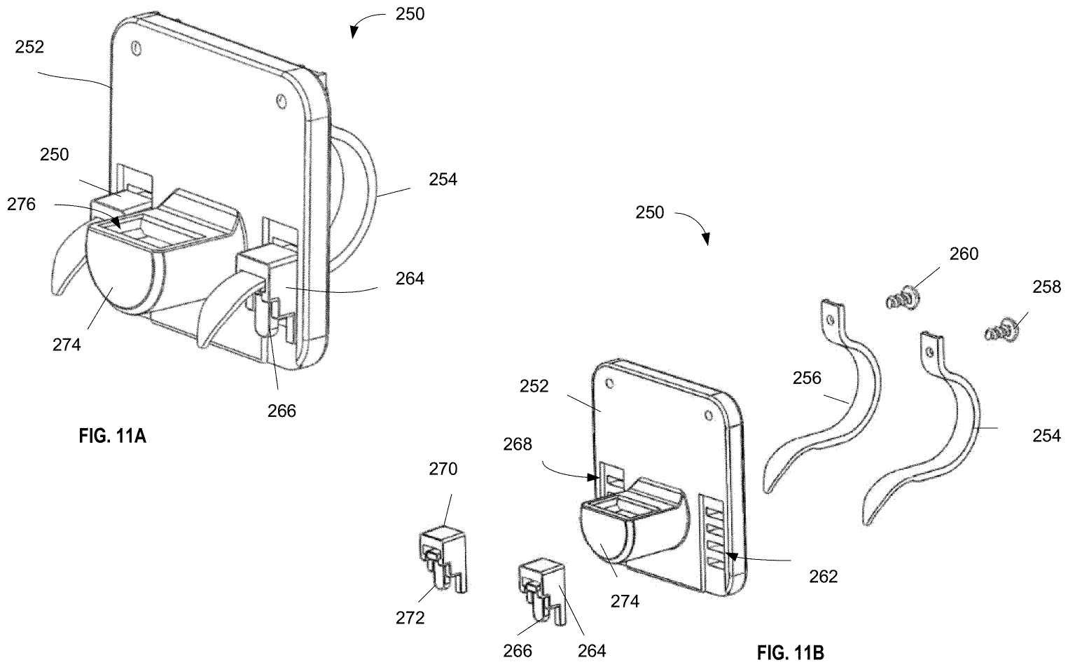

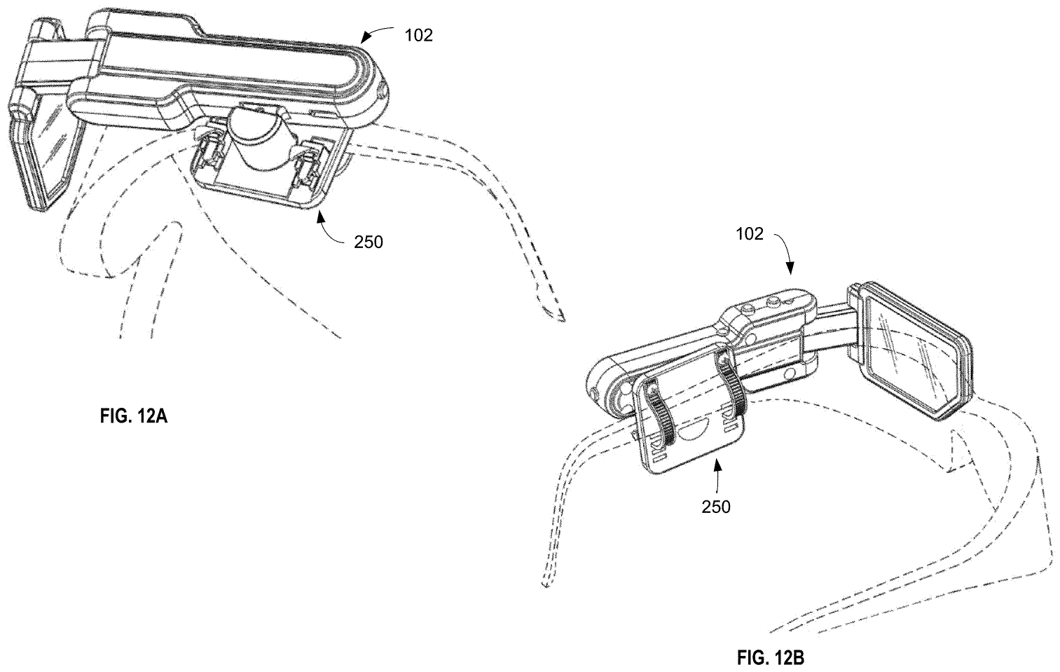

FIGS. 11A and 11B are perspective and exploded views of a mounting structure 250, in embodiments. FIGS. 12A and 12B are perspective views of mounting structure 250 and occluder 102 attached to a pair of glasses. FIGS. 11A, 11B, 12A and 12B are best viewed together in the following description.

Mounting structure 250 includes a plate 252 which is retained against an arm of a pair of glasses by zip-ties 254, 256. Zip-ties 254, 256 may be attached close to an upper edge of plate 252 by screws 258, 260, although any attachment means that securely fastens the zip-ties to the plate may be used. Zip-tie 254 is wrapped around the arm of the pair of glasses and inserted into one of four slots shown at 262. The appropriate slot is selected to provide a secure fit around the arm. After passing through one of slots 262, zip-tie 254 is inserted into quick release bracket 264. To remove mounting structure 250, quick release tab 266 may be pressed to allow removal of zip tie 254 from quick release bracket 264. Similarly, zip-tie 256 may be inserted through one of slots 268 into quick release bracket 270 and removed using quick release tab 272. As described above for mounting structure 104, cup 274 includes internal ridges 276 for securing pivot 120 of occluder 102 as shown in FIGS. 2, 12A and 12B.

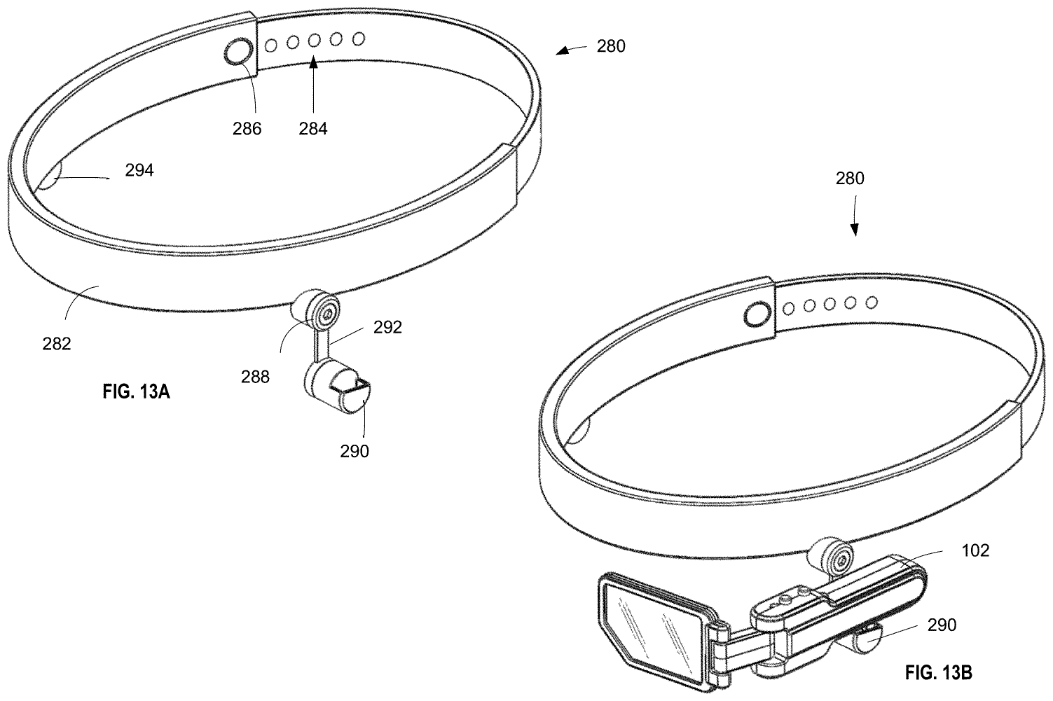

FIGS. 13A and 13B are perspective views of headband 280 for positioning occluder 102 of FIG. 2 in front of a user's eye without using glasses, in embodiments. Headband 280 includes band 282 which may be flexibly sized to fit across a user's forehead by means of tabs 284 and hole 286. Other mechanisms for allowing band 282 to accommodate different circumferences may be used. Mounting pivot 288 is attached to band 282 such that mounting pivot 288 would be generally adjacent to the user's temple when the headband is worn. Mounting pivot 288 is attached to cup 290 by arm 292. Cup 290 is similar to cup 274 of FIG. 11A, in embodiments. Cup 290 may be adjusted in a direction generally parallel to band 282 by rotating arm 292 around mounting pivot 288. As shown in FIG. 13B, occluder 102 may be inserted into cup 290 and adjusted for an optimal position as described above.

Headband 280 may be flexibly used to mount occluder 102 in front of either eye by means of mounting pivot 294, which is positioned across band 282 and generally adjacent to a user's opposite temple. Mounting pivot 294 is attached to an arm and cup (not shown) as shown and described for mounting pivot 288.

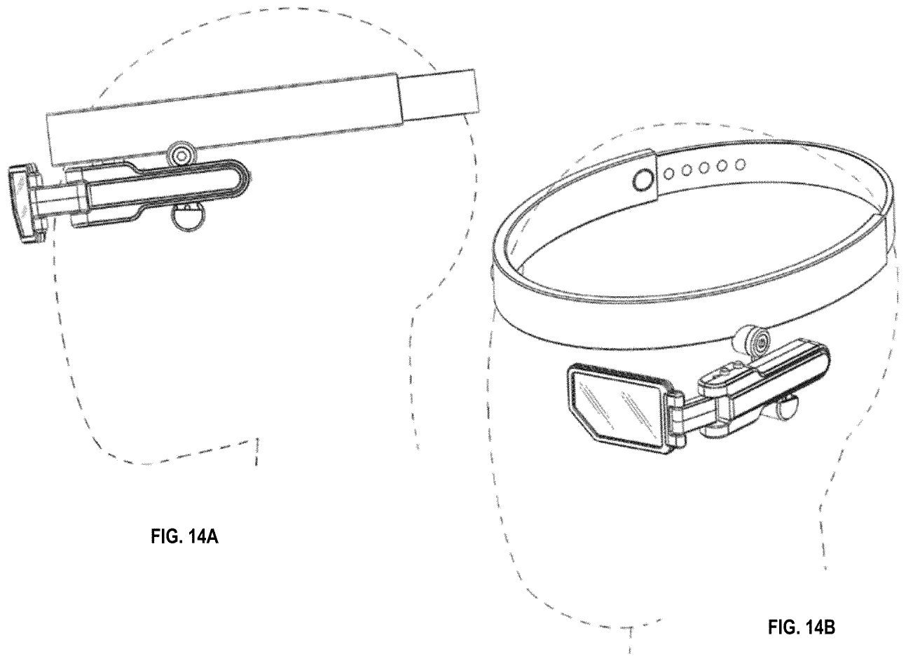

FIG. 14A is a side view of headband 280 and occluder 102 when headband 280 is worn. FIG. 14B is a perspective view of FIG. 14A, in embodiments. As explained for FIGS. 13A and 13B, headband 280 includes attachment mechanisms that allow occluder 102 to be worn in front of either eye.

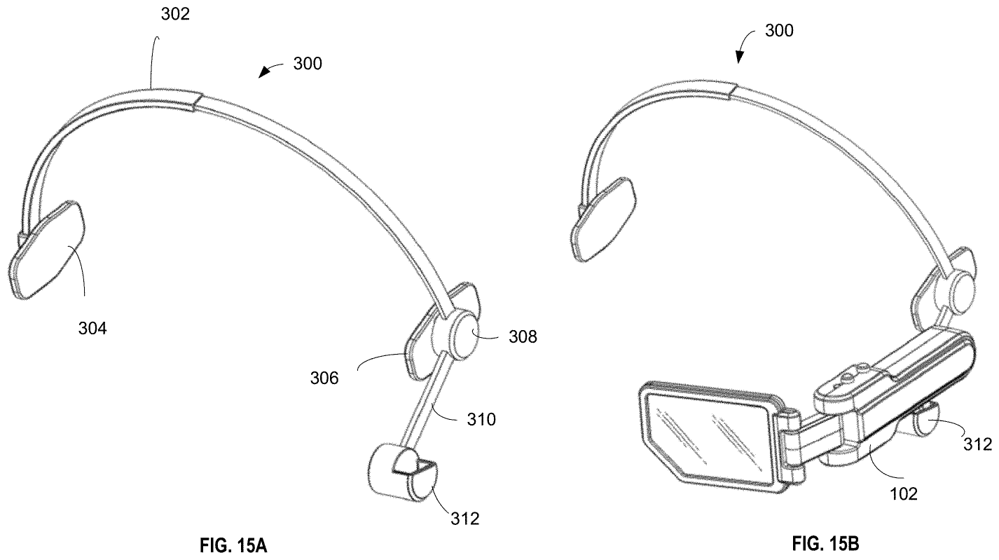

FIGS. 15A and 15B are perspective views of the occluder of FIG. 2 in use with headset 300, in embodiments. Headset 300 is adapted to be worn over the crown of a user's head, as shown in FIG. 17. Headset 300 includes an adjustable band 302 culminating in support structures 304 and 306 that retain headset 300 in position on the user's head.

Mounting pivot 308 is attached to cup 312 by arm 310. As described above for headband 280, arm 310 may rotate around mounting pivot 308 to provide flexible positioning of cup 312. FIG. 15B is a perspective view of headset 300 with occluder 102 attached to cup 312.

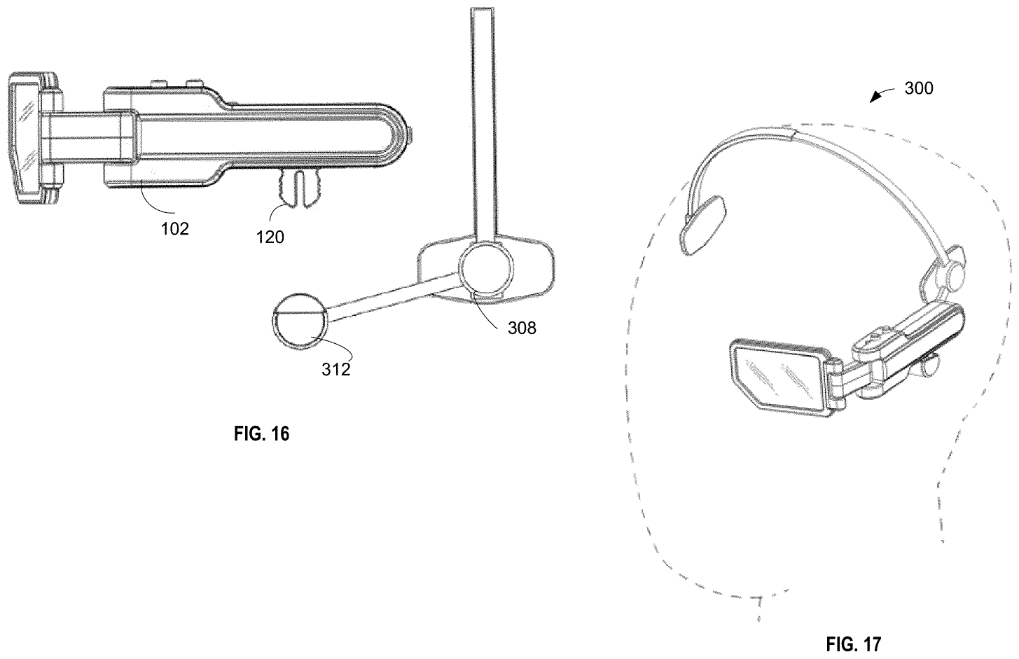

FIG. 16 is a side view illustrating positioning of occluder 102 prior to insertion of pivot 120 into cup 312. FIG. 17 is a perspective view of the headset of FIGS. 15A and 15B as it would be positioned on a user's head.

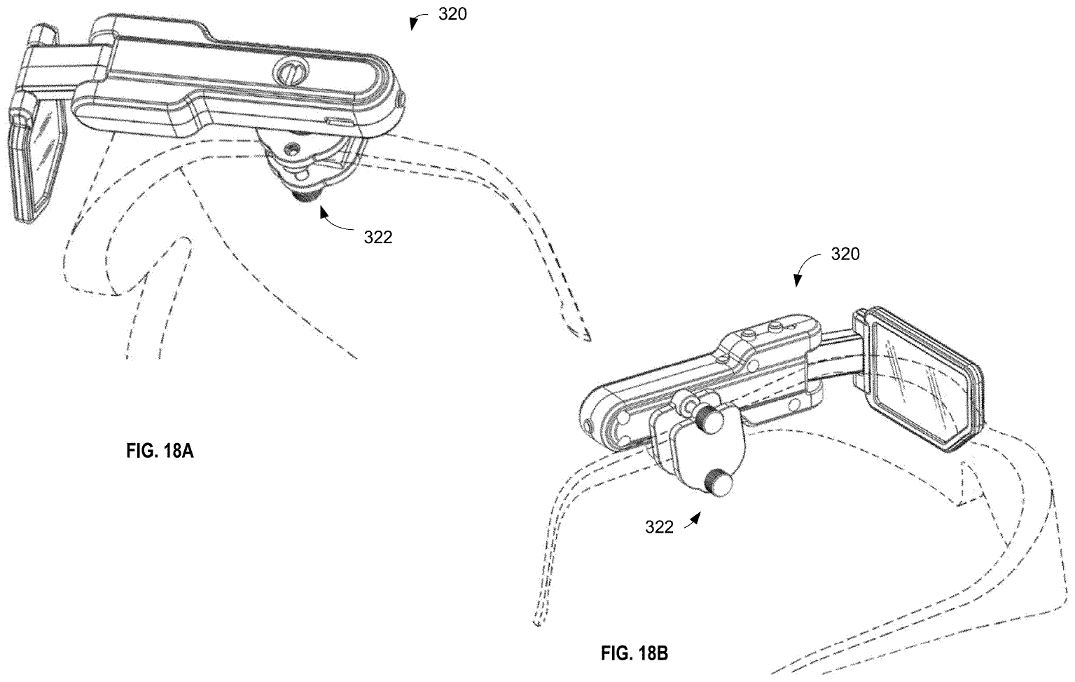

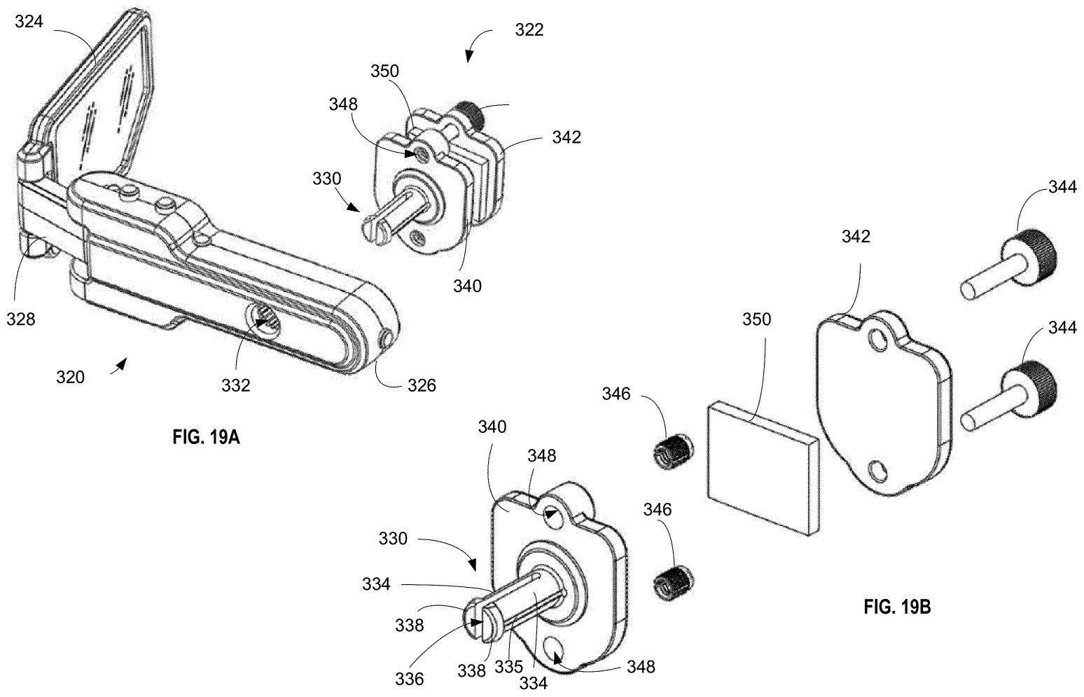

FIGS. 18A-18B are perspective views of another embodiment of an event-initiated occluding device including an occluder 320 mounted to a pair of glasses with a bayonet mounting structure 322, in embodiments. FIG. 19A is a perspective view of occluder 320 and bayonet mounting structure 322, in embodiments. FIG. 19B is an exploded view of bayonet mounting structure 322 of FIG. 19A, in embodiments. FIGS. 18A, 18B, 19A and 19B are best view together in the following description. In embodiments, although bayonet mounting structure 322 is shown attached to a left arm of a pair of glasses, it may also be attached to the right arm of a pair of glasses so that the lens of occluder 320 may be positioned in front of a user's right eye.

Occluder 320 is similar to occluder 102 in that lens 324 is attached to housing 326 by double hinge 328. Occluder 320 differs from occluder 102 in the mechanism of attaching to bayonet mounting structure 322. For example, occluder 320 does not include pivot 120. Further the housing and internal structure of occluder 320 are modified from that of occluder 102 to include opening 332. The functioning of occluder 320 is generally the same as that of occluder 102.

Bayonet mounting structure 322 includes a prong-like bayonet 330 that is inserted into opening 332 of occluder 320. Bayonet 330 includes two half stems 334 separated by gap 336, which allows half stems 334 to be compressed during insertion into opening 332 so as to ensure a snug fit. Each half stem 334 includes a longitudinal ridge 335 and culminates in a flange 338 at a distal end that cooperate with structures inside opening 332, as will be discussed in more detail below.

Bayonet 330 is mounted on outer face 340. Inner face 342 is attached to outer face 340 over an arm of a pair of glasses, as shown in FIGS. 18A-18B. Outer face 340 and inner face 342 may be flexibly attached over arms having varying thicknesses by means of screws inserted into nuts 346 that have been embedded in openings 348. A flexible material 350, for example, foam, may be mounted on one or both of outer face 340 or inner face 342 to help secure bayonet mounting structure 322 to the arm.

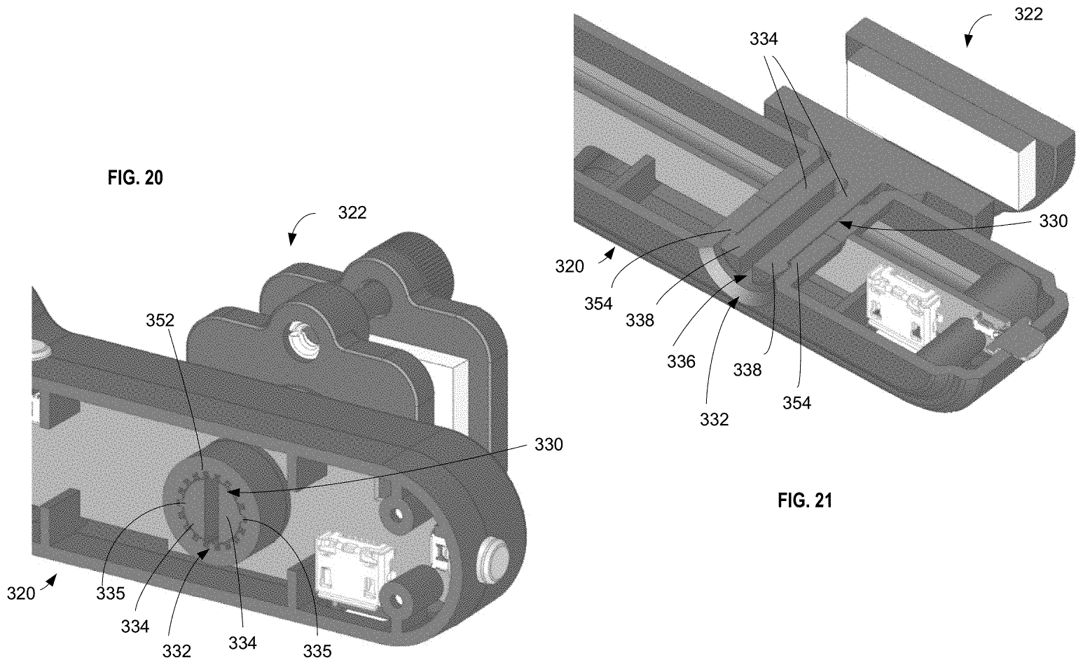

FIG. 20 is a horizontal cross-section of the occluder and mounting structure of FIG. 19A, in embodiments. Half stems 334 are visible inside opening 332. Longitudinal ridges 335 cooperate with ribs 352 around the circumference of opening 332 so that occluder 320 may be rotated around bayonet 330 and retained securely through 360 degrees of articulation. As occluder 320 is rotated longitudinal ridges snap into the space between adjacent ribs 352.

FIG. 21 is a vertical cross-section of the occluder and mounting structure of FIG. 19A, in embodiments. Half stems 334 culminate in flanges 338. Gap 336 between half stems 334 allows a slight compression as bayonet 330 is inserted into opening 332. When bayonet 330 is fully inserted, half stems 334 expand so that flanges 338 engage with surfaces 354 to retain occluder 320 on bayonet 330.

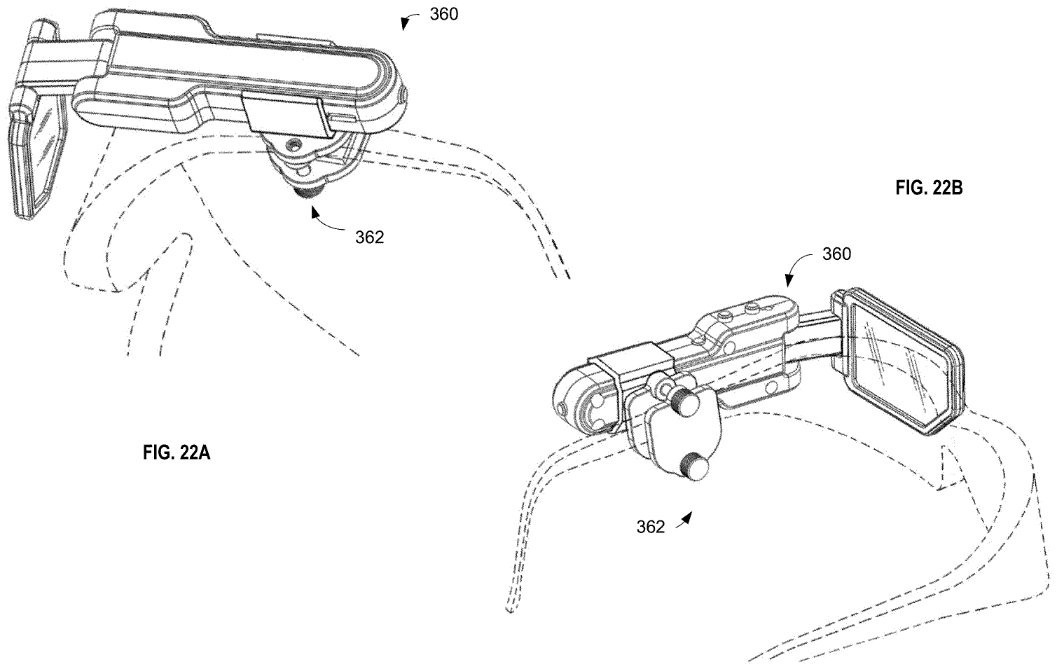

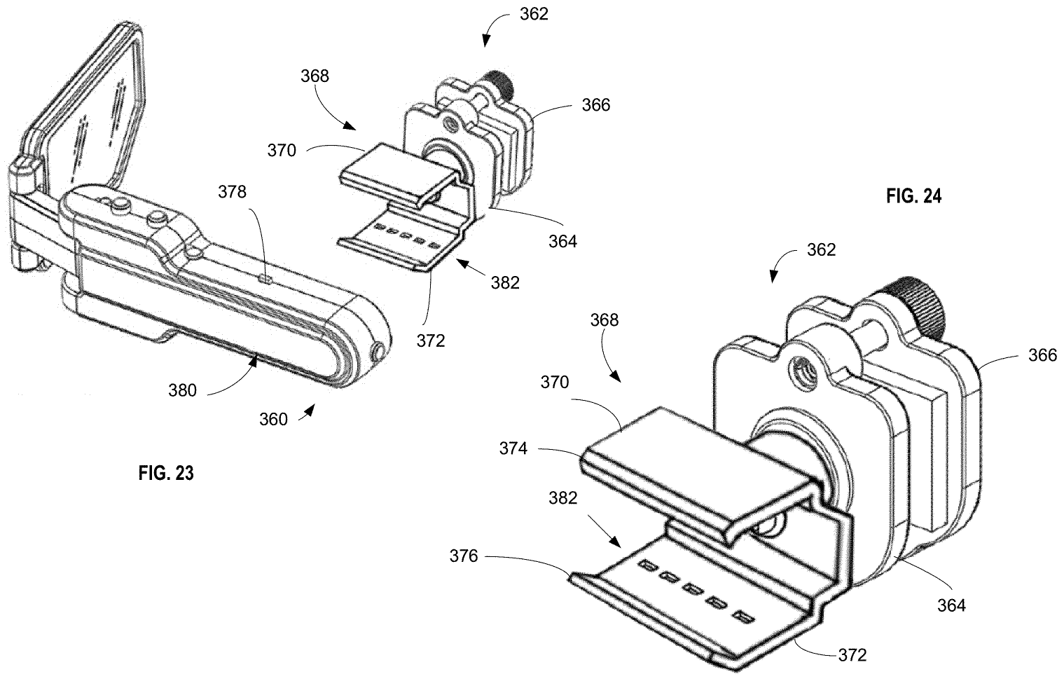

FIGS. 22A-22B are perspective views of an event-initiated occluding device mounted to a pair of glasses with a mounting structure, in embodiments. FIGS. 23 and 24 are perspective views of an occluder and corresponding mounting structure for FIGS. 22A-22B. FIGS. 22A, 22B, 23 and 24 are best viewed together in the following description.

Occluder 360 is generally similar to occluder 102 except that it does not include pivot 120. As shown in FIGS. 22A-22B, bayonet clip mounting structure 362 attaches to an arm of a pair of glasses similarly to bayonet mounting structure 322 of FIGS. 18A and 18B. In embodiments, although bayonet clip mounting structure 362 is shown attached to a left arm of a pair of glasses, it may also be attached to the right arm of a pair of glasses so that the lens of occluder 360 may be positioned in front of a user's right eye.

FIG. 23 is a perspective view of an occluder 360 and corresponding bayonet clip mounting structure 362 of FIGS. 22A-22B, in embodiments. Bayonet clip mounting structure 362 has an outer face 364 and inner face 366 that may be attached to either arm of a pair of glasses as described above for bayonet mounting structure 322. Instead of a bayonet, however, bayonet clip mounting structure 362 includes a clip 368 which is pivotably attached to outer face 364. Clip 368 includes upper arm 370 and lower arm 372 which are adapted to snap over occluder 360 as shown in FIGS. 22A-22B. Upper arm 370 includes downward angled front edge 374 and lower arm 372 includes an upward angled front edge 376 which engage with the housing of occluder 360 to provide a secure fit.

Clip 368 includes a row of detents 382 along lower arm 372. Corresponding detents (not shown) are located along upper arm 370. Detents 382 engage with tab 378 and 380 on occluder 360 so that occluder 360 may also be repositioned along clip 368 closer to or farther away from a user's eye.

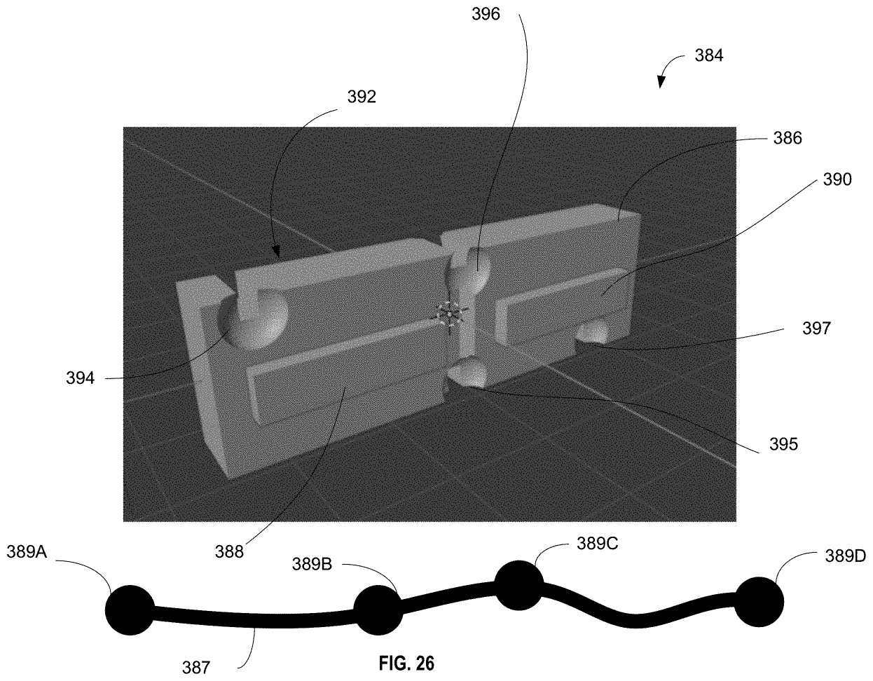

FIG. 26 is a perspective view of an elastic strap mounting structure 384, in embodiments. Block 386 is provided with magnets 388 and 390 for attaching to an occluder as described above. Inner side 392 of block 386, which is the back of block 386 as shown, may be attached to an arm of a pair of glasses by means of an elastic strap 387. In embodiments, the elastic strap may be a cord or strap that may be elongated, or stretched, along its length. A series of oblong or globe shapes 389A-D may be spaced along the elastic strap, each engaging with a recessed area in block 386. In embodiments, a first globe shape 389A may be nested in recessed area 394, then the elastic strap is wrapped around inner side 392 and the glasses arm, brought to the front of block 386 so that next globe shape 389B may be nested in recessed area 395. The elastic strap is then brought to the top of block 386 where globe shape 389C nests in recessed area 396, then again wrapped around the back of block 386 and the glasses arm. A final globe shape 389D is nested in recessed area 397. In embodiments, the elastic strap may be placed under tension while it is wrapped around block 386 so as to securely retain block 386 to the glasses arm. In further embodiments, any number of recessed areas and corresponding oblong or globe shapes may be provided.

In embodiments, any of occluders 102, 320 and 360 may be attached to a pair of glasses, a headband, a head set or any other type head gear such as a helmet. Further although several embodiments of mounting structures have been shown, others are contemplated, such as a mounting structure that incorporates one or more magnets in the occluder and a mounting structure so that the occluder is securely attached to the mounting structure. A mounting structure may be attached to any of the head gear above using any of the mechanisms described herein.

Any of the occluders described above may be controlled by one or more of several operating configurations, as described below.

Press and Hold Mode: When the user activates the occluder triggering mechanism, the occluder will darken the lens continuously during the period the occluder triggering mechanism remains activated by the user. When the user allows the triggering mechanism to return to its resting state, the occluder will no longer darken the lens.

ON/OFF Switch Mode: When the user activates the occluder triggering mechanism one time, a signal will be generated that causes the occluder to darken the lens. The occluder will then continue to darken the lens, without further intervention from the user, until such time as the user again activates the triggering mechanism. Upon the second activation of the triggering mechanism, the occluder will be turned off and the lens will return to its non-darkened state, remaining in that state until the user again activates the triggering mechanism.

Timed Interval Mode: When the user activates the occluder triggering mechanism one time, a signal will be sent to the occluder to darken the lens. The lens will then stay dark for a period of time preset by the user. At the end of the preset time period, the occluder will be turned off automatically, without further intervention from the user, and the lens will no longer be darkened.

Embodiments described herein may have a variety of advantages and practical applications, such as field shooting (e.g., hunting) or range shooting (e.g., target, trap, skeet, competition). Embodiments may be agnostic to the shooting implement, or they may be optimized to certain shooting implements, such as shotgun, rifle, pistol, bow, crossbow, or the like.

Further, related embodiments may be generally applicable in, or specifically adapted to, other sporting activities that involve aiming or hand-eye coordination, such as baseball, billiards, darts, golf, or the like. Other related applications or embodiments may include uses or adaptations for non-sporting aiming activities, such as surveying, telescopic or microscopic observation, use of a viewfinder or the like.

Many changes in the details, materials, and arrangement of parts and steps, herein described and illustrated, can be made by those skilled in the art in light of teachings contained hereinabove. It will be understood that certain features and sub-combinations are of utility and may be employed without reference to other features and sub combinations and are contemplated within the scope of the claims. Accordingly, it will be understood that the following claims are not to be limited to the embodiments disclosed herein and can include practices other than those specifically described, and are to be interpreted as broadly as allowed under the law. Additionally, not all steps listed in the various figures need be carried out in the specific order described.

We claim:

1. A wearable apparatus, comprising:
a lens controllable to selectively occlude vision;
a housing enclosing a controller and coupled to the lens, the housing adapted to be worn by a user so that the lens is positioned in front of a user's eye; and
a user-activated trigger mechanism adapted to transmitting a signal to the controller when activated by the user, the controller causing the lens to occlude the user's eye.

2. The wearable apparatus of claim 1, further comprising a mounting structure, said mounting structure adapted to be attached to a headband.

3. The wearable apparatus of claim 1, further comprising a mounting structure, said mounting structure adapted to be attached to a headset.

4. The wearable apparatus of claim 1, further comprising a mounting structure, said mounting structure adapted to be attached to a pair of glasses.

5. An occlusion system, comprising:
a lens controllable to temporarily occlude vision;
a housing;
a double hinge coupling the lens to the housing, the double hinge flexibly positioning the lens in front of a user's eye;
a mounting structure removably coupling the housing to a device worn on a head of the user;
a controller for receiving a signal to cause the lens to occlude a portion of a field of vision of the user's eye; and
a user-activated trigger mechanism comprising a sensor for detecting an action of the user, generating the signal to occlude vision and transmitting the signal to the controller.

6. The occlusion system of claim 5, wherein the signal is transmitted wirelessly.

7. The occlusion system of claim 5, wherein the mounting structure is adapted to attach the housing to either arm of a pair of glasses.

8. The occlusion system of claim 7, wherein the double hinge enables the lens to be oriented relative to a proximal lens of the pair of glasses.

9. The occlusion system of claim 5, wherein the lens is electrically or electromagnetically controlled to temporarily occlude vision.

10. The occlusion system of claim 9, wherein the lens comprises an electrically-controllable liquid crystal display (LCD).

11. The occlusion system of claim 9, wherein the lens comprises a microelectromechanical systems (MEMS) shutter array.

12. The occlusion system of claim 9, wherein the controller causes the lens to be occluded for a selectable degree of opacity.

13. The occlusion system of claim 9, wherein the controller causes the lens to be occluded for a selectable period of time.

14. The occlusion system of claim 13, wherein the controller causes the lens to be occluded after a selectable delay.

15. The occlusion system of claim 5, wherein the user-activated trigger mechanism further comprises a pressure sensor.

16. The occlusion system of claim 15, wherein the user-activated trigger mechanism further comprises a glove.

17. The occlusion system of claim 5, wherein the user-activated trigger mechanism further comprises a membrane switch.

18. An occluder for temporarily occluding a user's vision in one eye in response to a user-activated event when mounted to a device worn on a user's head, comprising:
an electrically-controllable lens;
a housing adapted to be mounted to the device so that the electrically-controllable lens is positioned in front of either of a user's eyes;
a double hinge coupling the electrically-controllable lens to the housing, the double hinge flexibly positioning the electrically-controllable lens in front of a user's eye;
a controller for receiving a signal to cause the electrically-controllable lens to occlude a portion of a field of vision of the user's eye.

19. The occluder of claim 18, wherein the signal is generated by a trigger activated by the user.

20. The occluder of claim 18, wherein the electrically-controllable lens further comprises a liquid crystal display (LCD).
